(19) United States
(12) Patent Application Publication
(10) Pub. No.: US 2016/0375240 A1
(43) Pub. Date: Dec. 29, 2016

(54) Method for iontophoretic body fat treatment and related apparatus

(71) Applicants: Jamal Yanaki, Salt Lake City, UT (US); Ralph Karl Koschinsky, Sandy, UT (US)

(72) Inventors: Jamal Yanaki, Salt Lake City, UT (US); Ralph Karl Koschinsky, Sandy, UT (US)

(73) Assignee: ActivaDerm, Inc. a Utah Corporation

(21) Appl. No.: 14/545,839

(22) Filed: Jun. 26, 2015

(51) Int. Cl. A61N 1/30; A61M 37/00; A61N 1/04

(52) U.S. Cl. CPC A61N 1/303; A61N 1/0428; A61M 37/00; A61M 2037/0007

(57) ABSTRACT

A roll of body wrap material is provided. The body wrap material may carry a medicament useful in addressing undesirable or unsightly human body fat. The body wrap material carries a conductive material used to establish an electric circuit for iontophoretic treatment. An area of a human body is wrapped with the body wrap material. An electric circuit is established from the medicament in the wrap, the conductive matter, human skin, and a power source. When the circuit is powered, ions of the medicament in the wrap are driven throught the human skin in the circuit in order to provide a treatment to the body fat beneath such skin.

### BACKGROUND

1. Field of the Invention

The invention disclosed herein relates to a method for transdermal administration of medicaments to human subjects for the purpose of providing a treatment related to the presence and/or appearance of the subject's body fat in the area of treatment. More particularly, the present invention pertains to active iontophoretic delivery methods, devices and systems in which electrical current is applied to for the purpose of driving medicament through the surface of the skin in order to provide such treatment. A roll or strip of medicament-carrying self-adhesive wrapping material can be wrapped around a body part where fat tissue is present. Electrodes can be attached and ionotophoresis can drive the medicament from the wrapping material into human tissue in order to provide a desired treatment for body fat found in the region of treatment.

2. Background Art

It is well-known that many persons feel self-conscious and even embarrassed by the appearance of fat tissue on their bodies. In particular, the appearance created by excess fat tissue, as well as the appearance of fat known as cellulite, are considered undesirable. Generally, cellulite is a term used to describe deposits of dimpled fat found on the thighs and buttocks, and is commonly found on women. It can also be found around the midsection and on arms. In a medical sense, cellulite consists of strands of fibrous tissue that connects the skin to deeper tissue layers along with separate compartments that contain fat cells. When fat cells increase in size, these compartments bulge and produce a waffled appearance of the skin which lay persons refer to as cellulite.

Although diet and exercise can tend to reduce overall fat content of a person's body, diet and exercise alone do not solve all issues relating to the appearance of body fat, so there is a strong demand for creams and medicaments which can be applied to the skin to reduce the amount of fat present, tone or tighten the skin in the area of undesirable body fat, firm up flabby areas, and/or conceal or otherwise improve the appearance of cellulite. Such medicaments include fat-dissolving cream, cellulite removing cream, and skin toning or tightening cream. The medicaments may address the fundamental problem of excess body fat by breaking down the fat so that it may be carried away. Or the medicaments may firm up the fat or the skin to improve the general appearance of that area of the body.

A wide variety of medicaments can be used to address body fat, and cellulite in particular. By way of example, herbal products, evening primrose oil, dried fucus vesiculosis extract, gelatine, fish oil, glycerol, soya oil, grape seed, bioflavonoids, soya lecithin, fatty acids, dried sweet clover extract, dried ginkgo biloba extract, clover extract, grape seed bioflavonoids, fucus vesiculosus extract, soya lecithin, and iron oxide, as well as many other chemicals, substances and compounds, may be used to address body fat

A particular problem in the prior art was that desired medicaments used to address body fat problems were placed on human skin in cream, gel or liquid form. Thos medicamanets were absorbed very slowly, if at all, and therefore had little beneficial effect on unwanted body fat.

Therefore, as exaplained in the Detailed Description below, the inventors have developed methods, systems and devices which use iontophoresis to drive a medicament into human tissue for the treatment of body fat.

During active iontophoresis, direct electrical current is used to cause ions of a medicament to move across the surface of the skin and to diffuse into underlying tissue. The surface of the skin is not broken by this administration of the medicament. When conducted within appropriate parameters, the sensations experienced by a subject during the delivery of the medicament in this manner are not unpleasant.

The direct current employed in active iontophoresis systems may be obtained from a variety of electrical power sources. These include consumable and rechargeable batteries, paired regions of contrasting galvanic materials that when coupled by a fluid medium produce minute electrical currents, and electrical equipment that ultimately receives power from a wall socket. The later in particular are of such bulk, weight, and cost as to necessitate being configured as items of equipment distinct from the electrical contacts that are applied directly to the skin in administering a medicament iontophoretically. Accordingly, such power sources limit the mobility of the patient during the time that treatment is in progress.

A flow of electrical current requires an uninterrupted, electrically-conductive pathway from the positive pole of a power source to the other, negative pole thereof. Living tissue is made up primarily of fluid and is, therefore, a conductor of electrical current. In an iontophoretic circuit, the opposite poles of a power source are electrically coupled to respective, separated contact locations on the skin of the subject. The difference in electrical potential created by the power source between those contact locations causes a movement of electrons and electrically charged molecules, or ions, through the tissue between the contact locations.

In an active iontophoretic delivery system, the polarity of the net overall electrical charge on dissolved molecules of a medicament determines the nature of the electrical interconnection that must be effected between the power source that is used to drive the system and the supply of medicament that is positioned on the skin of the patient at one of the contact locations to be used by the system. A positively charged medicament against the skin of a patient is coupled to the positive pole of the power source that is to be used to administer the medicament iontophoretically. Correspondingly, a negatively charged medicament on the skin of a patient must be coupled to the negative pole of such a power source. Examples of substances which may be mixed with medicaments in order to facilitate delivery by iontophoresis include bupivacaine hydrochloride, acetic acid, calcium chloride, betamethasone, sodium phosphate, lidocaine hydrochloride, copper sulfate, zinc chloride, dexamethasone, sodium phosphate, lidocaine fentinol, magnesium sulfate, naproxen, sodium chloride, sodium salicylate, ascorbic acid, hydroquinone, and vitamins A, C, D, or E or other vitamins. These and other substances driven through skin by iontophoresis can be considered a medicament.

The medicament is positioned electrically conductively engaging the skin of the subject at an anatomical location overlying the tissue to which the medicament is to be administered. The medicament matrix can take the form of a gel suspension of the medicament or in or on an absorbent or carrier material, such as gauze or cotton.

An iontophoretic circuit for driving the medicament through the unbroken skin is established by coupling the appropriate pole of the power source through the medicament matrix to the skin of the subject at the anatomical location at which the medicament is to be administered. Simultaneously, the other pole of the power source is coupled to an anatomical location on the skin of the subject that is distanced from the medicament matrix. The coupling of each pole of the power source is effected by the electrical connection of each pole to a respective electrode. The electrode at the medicament matrix is referred to as an active electrode; the electrode at the contact location on the skin distanced from the medicament matrix is referred to as a return electrode.

The medicament matrix with an associated active electrode may be conveniently retained against the skin at a first location on a roll or strip of material carrying the medicament, while the return electrode may be retained against the skin on a second location which is positioned some distance from the fist location.

The use of iontophoresis to administer medicaments to a subject is advantageous in several respects.

Medications delivered by an active iontophoretic system bypass the digestive system. This reduces digestive tract irritation. In many cases, medicaments administered orally are less potent than if administered transcutaneously. In compensation, it is often necessary in achieving a target effective dosage level to administer orally larger quantities of medicament than would be administered transcutaneously.

Active iontophoretic systems do not require intensive skin site sanitation to avoid infections. Patches and the other equipment used in active iontophoresis do not interact with bodily fluids and, accordingly, need not be disposed as hazardous biological materials following use. Being a noninvasive procedure, the administration of medicament using an active iontophoretic system does not cause tissue injury of the types observed with hypodermic injections and with intravenous catheterizations. Repeated needle punctures in a single anatomical region, or long term catheter residence, can adversely affect the health of surrounding tissue. Needle punctures and catheter implantations inherently involve the experience of some degree of pain. These unintended consequences of invasive transcutaneous medicament administration are particularly undesirable in an area of the body that, being already injured, is to be treated directly for that injury with a medicament. Such might be the case, for example, in the treatment of a strained muscle or tendon.

With some exceptions, no pharmacologically significant portion of a medicament delivered iontophoretically becomes systemically distributed. Rather, a medicament delivered iontophoretically remains localized in the tissue at the site of administration. This minimizes unwanted systemic side effects, reduces required dosages, and lightens the burdens imposed on the liver and kidneys in metabolizing the medicament.

The dosage of a medicament delivered iontophoretically is conveniently and accurately measured by monitoring the amount and the duration of the current flowing during the administration. With current being measured in amperes and time being measured in minutes, the dosage of medicament given transcutaneously is given in units of ampere-minutes. Due to the minute quantities of medicament required in active iontophoresis, medicament dosage in active iontophoresis is generally prescribed in milliamp-minutes. Dosage measured in this manner is more precise than is dosage measured as a fluid volume or as a numbers of tablets.

Finally, the successful operation of an active iontophoretic system is not reliant in any significant respect on the medical skills of nurses or doctors. Foregoing the involvement of such medical personnel in the administration of medicaments, whenever appropriate, favors the convenience of patients and reduces the costs associated with the delivery of such types of therapy.

Consequently, existing iontophoreis technology can be applied to novel methods, devices and systems in order to drive substances through human skin in order to address the unwanted or unsightly body fat.

### SUMMARY

The present invention promotes the wide use of active iontophoretic systems by providing improved components and combinations of components for active iontophoretic systems which can administer a medicament for the treatment of human body fat. The present invention allows a patient to wrap the affected area of the body with a medicament-carrying wrap, establish an electric circuit, and use iontophoresis to efficiently drive the medicament into the desired area of the human body. This method and system offers convenience, ease of use, efficiency and affordability in the treatment of unwanted or unsightly body fat.

Additional objects and advantages of the invention will be set forth in the Detailed Description which follows, and in part will be obvious from the description, or may be learned by the practice of the invention.

### BRIEF DESCRIPTION OF THE DRAWINGS

FIG. 1 depicts an example roll of iontophoretic wrap carrying a medicament intended to treat an area of human body fat.

FIG. 2 depicts a cross sectional view of the example wrap of FIG. 1 at 2-2.

FIG. 3 depicts the example wrap of FIG. 1 applied to an area of human body fat for the purpose of iontophoretically delivering a medicament to the area of body fat, which in this example is located on the thigh of a female subject.

### DETAILED DESCRIPTION

Referring to FIG. 1, a roll 101 of wrap material 102 which may carry be used go deliver a fat-treating medicament is depicted. The roll 101 is composed of a material 102 which can be dispensed from the roll to wrap around a body part which has body fat needing treatment. Material 102 from the roll 101 may be cut from the roll in strip or other form. Alternatively the material 102 may delivered in strip or patch form.

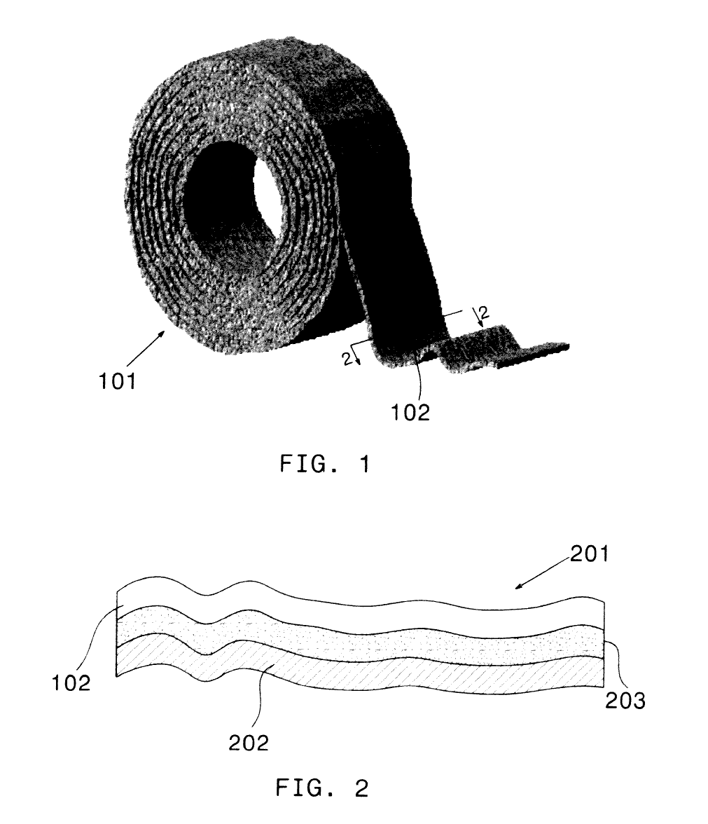

Referring to FIG. 2, an example cross-sectional view 201 of material 102 from roll 101 at 2-2 is provided. In this view, the material 102, a medicament 202, and a layer of conductive matter 203 are depicted as separate layers on the roll.

The conductive matter 203 may be applied to the roll of material 102 as a separate layer on the material. The conductive matter 203 could be sprayed onto the material 102, printed on the material 102, or otherwise applied to the material 102. Or the conductive mater 203 could be pressed into the material 102, diffused into the material 102, manufactured integral with the material 102, or otherwise carried by or in the material 102. Alternatiely the conductive matter could be applied directly to the material 102 or to a human body by the user rather than being manufactured with the roll of material 102.

The medicament 202 may is shown as being carried by the material 102 as a separate layer, thus yielding a roll with a layer of material, a layer of conductive matter and a layer of medicament. Alternatively, the medicament 202 may be infused into the material 102 rather than being a separate layer. Or the material and conductive matter can arrive at a patient's location without a medicament, and the medicament can be applied to the skin of a patient before the material 102 is placed on the patient. In that case, the material 102 with conductive matter forms three layers of material, conductive matter and medicament only when in place on a patient's body.

The conductive matter 203 can be used to establish an anode or cathode for completing the electrical circuit needed for iontophoretic treatment. In this example, the conductive matter is carbon which has been printed on the wrap material, although other conductive matter could be any matter capable of serving as an anode or athode. For ease of use, it is expected that many embodiments of the inventions disclosed herein will use conductive matter and medicament applied to the wrap material before the wrap is received by the patient so that the patient merely needs to apply the wrap to a body part, establish an electrical connection and turn the power on. Simplification of procedures to be performed by the patient can result in greater treatment success.

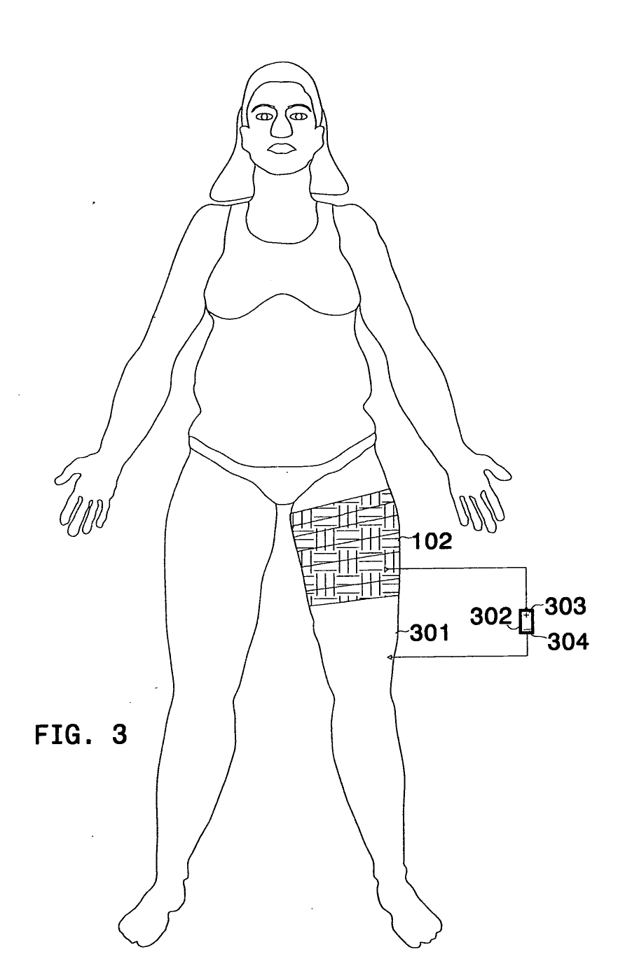

Referring to FIG. 3, wrap material 102 from a roll 101 (from FIG. 1) has been wrapped around a human body part 301 (human thigh in this example) in need of fat treatment by iontophoresis. A power source such as a battery 302 provides positive 303 and negative 304 poles which establish an electric circuit with the medicament and the patient's skin in order to drive ions of the medicament through the patient's skin into underlying tissue. When the wrap is in place over a fat deposit, the power source can be activated and a fat-treating medicament can be driven through the skin of a patient by iontophoresis over the course of several minutes. Thereafter the wrap can be removed and the used wrap can be discarded.

The medicament wrap used in the invention can take a variety of forms, and the example forms dsclosed herein are not intended to be limiting. For example, the wrap can be a stretchable or elastic self-adhesive material which when wrapped around an area of the human body will stick, adhere or affix to itself in order to remain position throughout the duration of iontophoretic treatment. The wrap can be porous for carrying medicament. The wrap can be fibrous, with the fibers having self-affinity so that the wrap may be secured to itself without separate fasteners. The wrap mey be stretchable or elastic, at least in part due to the fibers being elastic. Such a wrap may be stretched as it is wrapped around a portion of the human body so that it will of its own accord maintain pressure on the skin of the patient, thus keeping medicament-containing material in contact with the skin during iontophoresis. The material may be woven or non-woven as desired.

Although the medicament wrap is depicted above as coming in a roll, it could be presented in a sheet, in a strip, folded or crumpled.

The wrap is preferably stretchable so that it can easily conform to a human body part. The wrap may be elastic in nature, and use of a crinkle pattern in the wrap can provide increased loft and stretchability, as well as projecting fibers of the wrap for self-aherence.

The fibers of the wrap can be natural fibers such as cotton, flax, other vegetable fibers, animal hair or otherwise. The fibers of the wrap can also be man-made such as polyester, nylon, other polymers or other man-made fibers.

It is desirable for the medicament wrap to stick to itself but not to other objects, so that it will remain in place during iontophoresis without being pulled off or making a mess by adhering to other objects or substances.

Although iontophorsis has been described generally above, some additional information concerning implementation of iontophoresis in conjunction with a fat-treating medicament wrap may be helpful to the reader.

The medicament wrap in conjunction with a direct current power supply, a positive electrode electrically coupled to a pole of said power supply, a negative electrode coupled to the other pole of said power supply, and the patient's skin will create an electric circuit. Current can flow through that circuit and through the skin to create ions of medicament which are driven through the skin where they can provide a beneficial effect on the underlying human tissue.

When the above-described circuit has been established, the current electrically charges at least some molecules of the medicament and current flow drives at least some of said electrically charged medicament molecules of the medicament into the living skin which forms a part of the electric circuit. Electrical potential imposed across the skin in contact with medicament wrap produces electrical current flow by causing electrolysis in some molecules in the medicament. The living skin provides resistance for the electric circuit between positive electrode and negative electrodes in the circuit. The medicmant may contain water molecules and current flow electrolysis in the medicament. Electrolysis includes breaking a positively-charged hydrogen ion of a water molecule in said medicament from the water molecule to leave a negatively-charged hydroxyl radical of that same molecule. The hydrogen ion and said radical migrate in respective opposite directions through the medicament. The hydrogen ion moves toward a negative pole of the electric circuit of which the medicament is part, while said hydroxyl radical move toward a positive pole of said electric circuit, which in turn drives at least some of said medicament into the living skin of the electric circuit where the medicament may reach underlying fat deposits.

If desired, the fat treatment system herein may be presented as a kit. The kit can include a roll or strip of medicament-carrying body wrap, a power source such as a DC battery, and electrodes for using the battery and the wrap to establish an electric circuit with human skin in order to carry out iontophoretic delivery of the medicament in the wrap.

Alternatively, the invention herein can be considered a method. The method can include obtaining a length of medicament-containing wrap, the wrap having a layer of stretchable self-adhering material, a layer of conductive matter on the material, and a layer of medicament on the material. Then the wrap is wrapped around a part of a human body where fat is located. Positive and negative electrodes are established, a power source is turned on, and electrical current flows to the wrapped patient. Electrodes form an electric circuit with medicament on the wrap, conductive matter on the wrap, human skin, and the power source. Turning on the power source cause iontophoretic delivery of the medicament through the skin to fat deposits beneath the skin, and the medicament has a desired effect on human body fat in the area of treatment.

The medicament can include, if desired, a chemical from the group consisting of Bupivacaine hydrochloride, Calcium chloride, Lidocaine hydrochloride, Zinc chloride, and Lidocaine. The medicament can also include a chemical from the group consisting of Betamethasone sodium phosphate, Dexamethasone sodium phosphate, Fentinol, Copper sulfate, Acetic acid, Magnesium sulfate, Naproxen sodium, Sodium chloride, and Sodium salicylate.

The medicament used in this invention can include caffeine in order to take advantage of caffeine's ability to open capillaries and increase blood flow in the area of body fat treatment. Retinol may also be used in the medicament because it tends to encourage collagen production which makes skin thicker and tends to conceal underlying cellulite. Dimethylaminoethanol or DMAE can be included in the medicament. It is an antioxidant derived from fish that when combined with amino acids stimulates the muscles to contract and become firmer, thus reducing the flabby appearance of an area of human tissue. The medicament may also include cortisol or cortisol-management substances for their fat management effects. Without limiting the generality of the foregoing, and by way of example only, some fat tissue treatments may include medicaments which have a function corollary to fat removal may be utilized with the invented iontophoretic methods, devices and systems.

One preferred medicament composition is as follows:

| Medicament | % |
|---|---|
| Euglena Gracilis Extract | ≈50 |
| Water (Aqua) | qsp 100 |
| Caffeine | ≈1 |
| Glaucium Flavum Leaf | ≈0.1 |
| Extract | |

The example medicament may also include preservatives if desired such as sodium benzoate (0.5%) and potassium sorbate (0.3%). An antioxidant such as tocopherol (0.018%) can also optionally be included in the medicament.

An alternative medicament can include the active ingredient methylsilanol carboymethyl theophylline alginate (79.5%), water, butylene glycol (20%), sodium benzoate (0.5%) and any other desired ingredients.

The medicament may have the effect of promoting the unbinding of adipocyte from the extracellular matrix of adipose tissue through the stimulation of specific proteases involved in 3-dimensional tissue remodelling. Lipolysis in hypertrophied adipocytes in both monolayer culture and reconstructed hypodermis can occur. ATP re-synthesis can be achieved. Mitochondrial activity can be restored and an increase in cell energy can result. A decrease of triglyceride storage in mature adipocytes can be achieved with triglyceride stock reduction and adipocyte shedding. The medicament can prevent cell differentiation by activating fibronectin synthesis in preadipocytes and decrease the neoadipogenesis markers and lipid storage. The adipocytes reduce their lipid stock and reorganise their 3-dimensional conformation. The medicament can promote the unbinding of adipocytes from the extracellular matrix through the stimulation of specific proteases implicated in 3-dimensional tissue remodeling. The medicament can restore mitochondrial activity and stop oxidative stress involved in cellulite production. When driven into human fat tissue, the medicament can cause selective shedding of mature adipocytes and three-dimensional remodelling of hypertrophied adipose tissue. Consequently, a reduction in cellulite can occur and an improved physical appearance of the human subject can result.

In some medicaments, there can be an association of three active ingredients of vegetal origin: extract of glaucium flavum, extract of euglena gracilis and caffeine.

The medicaments inontophoretically delivered in the invention can achieve one or more of the following: (i) selective shedding of hypertrophied adipocytes from their substrate; (ii) reduction in the size of intermediate-size cells in order to prevent their growth by maintaining optimal lipolysis; (iii) prevention of the creation of new adipocytes; and (iv) restoration of mitochondrial metabolism by halting oxidative stress and retriggering ATP synthesis.

For more information on possible fat-treating medicaments, the reader is directed to World Intellectual Property Organization patent numbers WO 2006/075311, WO 2007/077541 and WO 2004/024695, each of which is hereby incorporated by reference in its entirety.

While the present invention has been illustrated and described with respect to a number of specific example embodiments, those skilled in the art will appreciate that variations and modifications may be made without departing from the fundamental principles of the invention as herein described, illustrated and claimed. The present invention may be implemented in a variety of different forms without departing from its fundamental characteristics or spirit. The described embodiments are to be considered as illustrative and are in no way intended to be restrictive of the scope of the invention. The scope of the invention is defined by the appending claims rather than the foregoing description. All changes which come within the meaning and range of equivalency of the claim are to be embraced within their scope.

1. A method for providing a thereapeutic treatment for undesirable or unsightly human body fat comprising the steps of:
selecting an area of a human body containing human body fat to be treated,
obtaining a length of iontophoretic body wrap material,
said iontophoretic body wrap material carrying a conductive matter for establishing an electrical contact, and
said inotophoretic body wrap material containing a medicament, said medicament being a fat-treating medicament that is useful for providing a treatment to human body fat,
wrapping said area of a human body with at least some of said iontophoretic body wrap material,
obtaining a power source,
obtaining electrodes,
connecting said power source and said electrodes to said iontophoretic body wrap material conductive matter and to human skin of a human body to establish an electric circuit, and
powering said electric circuit to inotophoretically drive at least some of said medicament through said human skin so that it can provide a treatment to human body fat beneath said skin.

2. A method as recited in claim 1 wherein said body wrap material includes elastic fibers so that said body wrap material is stretchable.

3. A method as recited in claim 1 wherein said body wrap material has a self-affinity so that it may be attached to itself without the use of separate fastening devices.

4. A method as recited in claim 1 further comprising the step of attaching said body wrap to itself without the use of separate fastening devices.

5. A method as recited in claim 1 wherein said wrap is porous for carrying said medicament.

6. A method as recited in claim 1 wherein powering said electric circuit creates a current electrically charges at least some molecules of said medicament in order to drive at least some of the into said skin.

7. A method as recited in claim 6 wherein electrical potential imposed across skin in contact with medicament wrap in said electric circuit produces electrical current flow by causing electrolysis of some molecules in said medicament.

8. A method as recited in claim 7 wherein said skin in said electric circuit provides resistance in said electric circuit between positive electrode and negative electrodes in the circuit.

9. A method as recited in claim 8 further comprising the step of performing electrolysis of said medicament to create positively-charged hydrogen and negatively-charged hydroxyl radicals which migrate in respective opposite directions through the medicament, which in turn drives at least some of said medicament into said human skin where the medicament may reach underlying human body fat to provide a treatment to said fat.

10. A method as recited in claim 9 wherein said conductive matter is carbon presented as a layer on said wrap.

11. An iontophoretic human body fat treatment kit comprising:
a roll of body wrap material,
a medicament carried by said body wrap material,
a conductive matter carried by said body wrap material,
a power source for powering inotophoresis,
a positive electrode for establishing an electric circuit with said conductive matter, said medicament in said body wrap, said power source, and human skin, and
a negative electrode for establishing an electric circuit with said conductive matter, said medicament in said body wrap, said power source, and human skin.

12. A kit as recited in claim 11 wherein said medicament can be driven across human skin by iontophoresis in order to provide a treatment effect on human body fat located beneath such skin.

13. A kit as recited in claim 12 wherein said wrap is elastic.

14. A kit as recited in claim 13 wherein said wrap has a self-affinity which causes said wrap to stick to itself so that it may be secured to a human body without the need for additional fastening devices.

15. A kit as recited in claim 14 wherein said conductive matter is carbon presented as a layer on said wrap.

16. A kit as recited in claim 11 wherein said conductive matter is carbon printed on said wrap material.

17. A kit as recited in claim 11 wherein said medicament is selected from the group consisting of:
euglena gracilis extract,
(ii) methylsilanol carboymethyl theophylline alginate, and
(iii) glaucium flavum extract.

18. A kit as recited in claim 11 wherein intophoretic delivery of said medicament to human fat tissue achieves at least one effect from the group consisting of: (i) selective shedding of hypertrophied adipocytes from their substrate; (ii) reduction in the size of intermediate-size cells in order to prevent their growth by maintaining optimal lipolysis; (iii) prevention of the creation of new adipocytes; and (iv) restoration of mitochondrial metabolism by halting oxidative stress and retriggering ATP synthesis.

19. A method for providing a thereapeutic treatment for undesirable or unsightly human body fat comprising the steps of:
selecting an area of a human body containing human body fat to be treated, obtaining a length of iontophoretic body wrap material,
said iontophoretic body wrap material carrying a conductive matter for establishing an electrical contact,
applying a medicament which has a fat-treating quality to said selected area of a human body containing body fat,
applying said length of wrap material over said medicament on said selected area of a human body,
obtaining a power source,
obtaining electrodes,
connecting said power source and said electrodes to said iontophoretic body wrap material conductive matter and to human skin of a human body to establish an electric circuit with said medicament, and
powering said electric circuit to inotophoretically drive at least some of said medicament through said human skin so that it can provide a treatment to human body fat beneath said skin.
